(19) United States
(12) Patent Application Publication
(10) Pub. No.: US 2010/0034832 A1
(43) Pub. Date: Feb. 11, 2010

(54) METHODS FOR MANIPULATING STEM CELLS

(75) Inventors: David T. Scadden, Weston, MA (US); Yoriko Saito, Kanagawa (JP); Eyal Attar, West Newton, MA (US)

Correspondence Address: EDWARDS ANGELL PALMER & DODGE LLP, P.O. BOX 55874, BOSTON, MA, 02205, US

(73) Assignee: THE GENERAL HOSPITAL CORPORATION, Boston, MA (US)

(21) Appl. No.: 12/304,667

(22) PCT Filed: Jun. 12, 2007
(86) PCT No.: PCT/US07/13824

§ 371 Date: Oct. 20, 2009

Related U.S. Application Data
(60) Provisional application No. 60/813,274, filed on Jun. 12, 2006.

(51) Int. Cl. A61K 39/395; A61K 35/12; C12Q 1/02; C12N 5/074; A61K 31/7088; A61K 31/7105

(52) U.S. Cl. 424/172.1; 424/93.7; 435/29; 435/325; 435/375; 514/44 A

(57) ABSTRACT

The invention generally features methods and compositions for enhancing stem cell function. In particular, the invention provides therapeutic or prophylactic methods that can increase survival, growth or proliferation during blood and/or stem cell transplant and protect stem cells in settings of injury.

This application claims priority to U.S. Application Ser. No. 60/813,274, filed on Jun. 12, 2006, the contents of which are incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference, and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference.

### STATEMENT OF RIGHTS TO INVENTIONS MADE UNDER FEDERALLY SPONSORED RESEARCH

This work was supported by the following grants from the National Institutes of Health, Grant Nos: R01 DK50234 and HL70989. The government may have certain rights in the invention.

### BACKGROUND OF THE INVENTION

Most chemotherapeutic agents kill cancer cells that are actively multiplying. Adverse side effects associated with chemotherapy are due to the toxic effects of such agents on healthy stem cells, including stem cells of the bone marrow that are largely responsible for generating white and red blood cells. Low white blood cell count (also called granulocytopenia or neutropenia) is a major dose-limiting factor with chemotherapy and is the cause for the most serious side effect of chemotherapy-infection. A low red blood cell count, or anemia, can also be a significant source of concern for patients receiving chemotherapy. A low platelet count, also called thrombocytopenia, is another dose-limiting factor with chemotherapy and is the cause for a serious side effect of chemotherapy-bleeding. Neutropenia and thrombocytopenia can delay chemotherapy, cause dosage reductions, or even cause changes in drug therapy because of drastic reductions in the stem cells that give rise to cells of the hematopoietic lineage. Methods for protecting stem cells from damage are urgently required.

P2 receptors are functionally diverse cell surface receptors that bind nucleotides adenine (ADP, ATP) and uridine (UDP, UTP). The P2 family of receptors can be subdivided into P2X receptors that are ionotropic ligand-gated ion channels and P2Y receptors that are metabotropic G protein-coupled seven-transmembrane receptors. Functionally, P2Y receptors have been found to participate in vascular and immune responses to injury. P2Y₁₄ was originally cloned from human immature myeloid cell line, KG1, and homologous molecules have since been identified in the rat and the mouse. The cloning of P2Y₁₄ from quiescent primary human bone marrow (BM) hematopoietic stem cells (HSCs) was previously reported, defining its function in bone marrow hematopoietic stem cell homing (Lee, B. C. et al. Genes Dev. 17, 1592-1604 (2003). The role of the P2Y₁₄ receptor in modulating stem cell response to injury was previously unknown.

### SUMMARY OF THE INVENTION

The invention is derived, in part, from the discovery that P2Y₁₄ receptor activation impedes the protection of stem cells post-injury. As described below, the present invention features methods and compositions for enhancing stem cell survival, growth, proliferation and expansion.

In one aspect, the invention provides a method for increasing stem cell expansion, the method comprising contacting a stem cell with an effective amount of an agent that inhibits P2Y14 receptor expression or biological activity, thereby increasing stem cell expansion.

Stem cells of the invention include, but are not limited to, mesenchymal, skin, neural, intestinal, liver, cardiac, prostate, mammary, kidney, pancreatic, retinal and lung stem cells.

In one embodiment, the stem cell is contacted in vivo or ex vivo.

In another embodiment, the stem cell is contacted in vivo in a subject having received a stem cell insult.

In yet another embodiment, the agent is an inhibitory nucleic acid molecule that decreases the expression of a P2Y14 receptor polynucleotide or polypeptide.

In a specific embodiment, the inhibitory nucleic acid molecule is an antisense molecule, short interfering RNA (siRNA), or short hairpin RNA (shRNA).

In yet another embodiment, the agent is an antibody that inhibits P2Y14 receptor activation.

In a specific embodiment, the antibody blocks the binding of a ligand to the receptor.

In another aspect, the invention provides a method for treating blood cell injury in a subject, the method comprising contacting a hematopoietic stem cell of the subject with an effective amount of an agent that inhibits the expression or biological activity of a P2Y14 receptor in the subject following an insult to a hematopoietic stem cell, thereby treating blood cell injury in the subject.

In one embodiment, the agent is an antibody that inhibits P2Y14 receptor activation.

In a specific embodiment, the antibody blocks the binding of a ligand to the receptor.

In another embodiment, the insult is administration of a chemotherapeutic agent.

In yet another embodiment, the agent is an inhibitory nucleic acid molecule that decreases the expression of a P2Y14 receptor polynucleotide or polypeptide.

In a specific embodiment, the inhibitory nucleic acid molecule is an antisense molecule, short interfering RNA (siRNA), or short hairpin RNA (shRNA).

In yet another embodiment, the agent is a small molecule.

In yet another aspect, the invention provides a method for increasing the amount of blood cells in a subject in need thereof, the method comprising administering to the subject an effective amount of an agent that inhibits the expression or biological activity of a P2Y14 receptor, thereby increasing the amount of blood cells in the subject.

In one embodiment, the agent is an antibody that inhibits P2Y14 receptor activation.

In a specific embodiment, the antibody blocks the binding of a ligand to the receptor.

In another embodiment, the subject has received an insult to a hematopoietic stem cell.

In yet another embodiment, the agent is an inhibitory nucleic acid molecule that decreases the expression of a P2Y14 receptor polynucleotide or polypeptide.

In a specific embodiment, inhibitory nucleic acid molecule is an antisense molecule, short interfering RNA (siRNA), or short hairpin RNA (shRNA).

In yet another embodiment, the agent is a small molecule.

In yet another embodiment, the subject has abnormal cells in bone marrow.

In yet another embodiment, the subject is diagnosed as having leukemia or myelodysplasia.

In yet another aspect, the invention provides a method of increasing stem cell survival, growth or proliferation the method comprising contacting a stem cell or progenitor cell that expresses a P2Y14 receptor with an effective amount of an agent that inhibits P2Y14 receptor expression or biological activity thereby increasing stem cell survival or proliferation. The method may further comprise growing the stem cell or stem cell progenitor.

In a specific embodiment, the progenitor cell is a hematopoietic progenitor cell and/or the stem cell is a hematopoietic stem cell. In one embodiment, the proliferation is by stem cell self-renewal.

In another embodiment, the stem cell is contacted in vivo in a subject having received a stem cell insult.

In yet another embodiment, the hematopoietic stem cell is in the bone marrow.

In yet another embodiment, the agent is an inhibitory nucleic acid molecule that decreases the expression of a P2Y14 receptor polynucleotide or polypeptide.

In a specific embodiment, the inhibitory nucleic acid molecule is an antisense molecule, short interfering RNA (siRNA), or short hairpin RNA (shRNA).

In various embodiments, the agent that inhibits P2Y14 receptor expression reduces P2Y14 receptor transcription, and/or reduces P2Y14 receptor translation.

In various embodiments, the agent that inhibits P2Y14 biological activity inhibits P2Y14 receptor activation. Biological activity can be monitored by measuring calcium influx, by measuring ligand binding, or by measuring hematopoietic stem cell function. The ligand can be, for example, uridine diphosphoglucose-glucose or another UDP-sugar.

In yet another aspect, the invention provides a method of increasing the number of self-renewing stem cells in a subject in need thereof, the method comprising the steps of: contacting an isolated population of cells comprising stem cells with a P2Y14 receptor inhibitor; and administering the cells to the subject, thereby increasing the amount of self-renewing stem cells in the subject. The isolated population of cells can be obtained from the subject (e.g., a human subject).

In one embodiment, the isolated population of cells are administered to the subject during a bone marrow transplant.

In another embodiment, the isolated population of cells are obtained from bone marrow.

In a specific embodiment, the bone marrow cells comprise a Lin⁻ cKit⁺Sca1⁺.

In yet another embodiment, the stem cells are hematopoietic stem cells.

In yet another aspect, the invention provides a method of increasing engraftment of a stem cell in a tissue of a subject in need thereof, the method comprising:

(a) contacting the stem cell with an agent that inhibits the expression or biological activity of a P2Y14 receptor; and

(b) providing the stem cell to a tissue, thereby increasing engraftment of the stem cell in the tissue.

In one embodiment, the stem cell is contacted in vivo in a subject having received a stem cell insult.

In various embodiments, methods of the invention may further comprise obtaining the agent or inhibitor.

In yet another aspect, the invention provides a method of identifying a candidate compound that promotes stem cell survival, growth or proliferation, the method comprising: a) contacting a cell that expresses a P2Y14 receptor with a candidate compound; and b) detecting a decrease in OPN expression or activity, wherein the decrease identifies a candidate compound that promotes stem cell survival or proliferation. The method may further comprise the step of identifying an increase in stem cell number.

In yet another aspect, the invention provides an isolated bone marrow derived cell comprising a P2Y14 receptor inhibitory nucleic acid molecule, wherein the P2Y14 receptor inhibitory nucleic acid molecule reduces expression of the P2Y14 receptor in the cell.

In one embodiment, the P2Y14 receptor inhibitory nucleic acid molecule is an siRNA, shRNA, or antisense RNA.

In yet another aspect, the invention provides a kit for promoting stem cell survival, growth, or proliferation comprising a P2Y14 receptor inhibitor, and instructions for using the inhibitor to promote stem cell survival, growth, or proliferation.

In yet another aspect, the invention provides a kit for increasing stem cell expansion comprising an agent that inhibits the P2Y14 receptor expression of biological activity and instructions for using the agent to increase stem cell expansion.

Other features and advantages of the invention will be apparent from the detailed description, and from the claims.

### BRIEF DESCRIPTION OF THE FIGURES

Further objects, features and advantages of the invention will become apparent from the following detailed description taken in conjunction with the accompanying figures showing illustrative embodiments of the invention, in which:

FIG. 1 depicts P2Y₁₄ protection of primitive hematopoietic stem cells following chemical injury through induction of relative quiescence and protection from apoptosis. At day 4 after intraperitoneal injection of 200 mg/kg cyclophosphamide (CTX), BM mononuclear cell % LKS⁺ (a) and % CD34^{lo/-}LKS⁺ (b) were measured. The apoptotic fraction of LKS⁺ (c) and CD34^{lo/-}LKS⁺ (d) cells were also quantified, showing greater extent of apoptosis in P2Y₁₄^{-/-} primitive HSCs following CTX-mediated BM injury. After a single intraperitoneal injection of 150 mg/kg 5-fluorouracil (5FU) at day 0 (arrows), PB leucocyte number (e) and PB Gr-1⁺ leucocyte number (f) were measured weekly for 9 weeks, showing greater than normal granulocyte count rebound response that persists for up to 9 weeks during recovery of peripheral counts following 5FU. LKS⁺ cells from P2Y₁₄^{-/-} and ^{+/+}BM were sorted and cultured in vitro in cell cycle-promoting cytokines for 3 days, followed by addition of 100 mM UDP-glucose into culture for 16 hours. The cells were then pulsed with BrDU for 45 minutes and BrDU incorporation was measured at 6 and 24 hours post-pulse (g), showing that UDP-glucose slows down cell cycle progression of P2Y₁₄^{+/+} primitive HSCs.

FIG. 2 depicts superior engraftment potential and self-renewal capacity of P2Y₁₄^{-/-} WBM. CD45.2⁺ whole BM mononuclear cells from each genotype were injected intravenously into lethally irradiated female CD45.1 recipients with equal numbers of whole BM mononuclear cells from male CD45.1 competitors. In primary (a), secondary (b) and tertiary (c) recipients, transplantation of P2Y₁₄^{-/-} whole BM cells resulted in greater PB % CD45.2 mononuclear cells, indicating superior engraftment and self-renewal capacities of P2Y₁₄^{-/-} HSCs. Limiting dilution competitive serial transplantations were performed using 1:1, 1:2 and 1:4 ratio of CD45.2+P2Y₁₄^{-/-} or ^{+/+} whole BM to CD45.1⁺ whole BM cells (d, e). In both primary (d) and secondary (e) recipients, P2Y₁₄^{-/-} whole BM showed statistically significantly greater competitive repopulating unit (CRU) equivalents compared with ^{+/+}.

FIG. 3 depicts the proposed role of P2Y₁₄ in BM response to injury. The proposed roles of P2Y₁₄ in acute (a) and recovery (b) phase of chemical injury to both BM hematopoietic cells and BM microenvironment, and in the setting of HSC transplantation (c) where only BM microenvironment is injured are illustrated.

### DETAILED DESCRIPTION OF THE INVENTION

### Definitions

By “agent” is meant any antibody, nucleic acid molecule, or polypeptide, or fragments thereof as well as chemical compounds, such as steroid or small molecule compounds.

By “allogeneic” is meant cells of the same species.

By “anti-sense” is meant a nucleic acid sequence, regardless of length, that is complementary to the coding strand or mRNA of a nucleic acid sequence. In one embodiment, an antisense RNA is introduced to an individual cell, tissue, organ, or to a whole animals. The anti-sense nucleic acid may contain a modified backbone, for example, phosphorothioate, phosphorodithioate, or other modified backbones known in the art, or may contain non-natural internucleoside linkages. Modified nucleic acids and nucleic acid analogs are described, for example, in U.S. Patent Publication No. 20030190659.

By “antibody” is meant any immunoglobulin polypeptide, or fragment thereof, having immunogen binding ability.

By “autologous” is meant cells from the same subject.

By “bone marrow derived cell” is meant any cell type that naturally occurs in bone marrow.

By “blood cell injury” is meant any disruption of the survival, growth, or proliferation of a red or white blood cell, blood progenitor cell or blood stem cell (e.g., a hematopoietic stem cell).

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “double stranded RNA’ is meant a complementary pair of sense and antisense RNAs regardless of length. In one embodiment, these dsRNAs are introduced to an individual cell, tissue, organ, or to a whole animals. For example, they may be introduced systemically via the bloodstream. Desirably, the double stranded RNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more. The dsRNA may contain a modified backbone, for example, phosphorothioate, phosphorodithioate, or other modified backbones known in the art, or may contain non-natural internucleoside linkages.

By “effective amount” is meant either the amount of P2Y14 receptor inhibitor or stem cells treated with a P2Y14 receptor inhibitor that either alone or together with further doses produce the desired therapeutic response (i.e., enhancing survival, growth or proliferation of stem cells).

By “engraft” or “engraftment” is meant the process of stem cell incorporation into a tissue of interest in vivo through contact with existing cells of the tissue.

By “expansion” is meant the propagation of a cell or cells without terminal differentiation.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids

By “hematopoietic progenitor cell” is meant a multipotent cell which has the potential to become committed to the hematopoietic lineage.

By “hematopoietic stem cell” is meant a pluripotent cell which has the potential to become committed to multiple lineages, including the hematopoietic lineage.

By “inhibitory nucleic acid” is meant a double-stranded RNA, siRNA, shRNA, or antisense RNA, or a portion thereof, or a mimetic thereof, that when administered to a mammalian cell results in a decrease in the expression of a target gene. Typically, a nucleic acid inhibitor comprises at least a portion of a target nucleic acid molecule, or an ortholog thereof, or comprises at least a portion of the complementary strand of a target nucleic acid molecule. Typically, expression of a target gene is reduced by 10%, 25%, 50%, 75%, or even 90-100%.

By “insult” is meant any natural or artificial (e.g., chemical) damage inflicted upon a cell.

By “insult to a hematopoietic stem cell” is meant any disruption of the normal functioning of the hematopoietic stem cell. Disruptions to hematopoietic stem cell function include, but are not limited to, decreases in the survival, growth, or proliferation.

By “isolated” is meant a material that is free to varying degrees from components which normally accompany it as found in its native state. “Isolate” denotes a degree of separation from original source or surroundings.

By “obtain” is meant purchasing, synthesizing, or otherwise acquiring.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “neoplasia” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells).

By “normal blood cell amount” is meant the average blood cell count of a healthy control subject.

By “P2Y14 polynucleotide” is meant a nucleic acid sequence encoding the P2Y14 polypeptide. Exemplary nucleic acid sequences include Genbank Accession No. D13631.

By “P2Y14 receptor” is meant an amino acid sequence having at least 85% or greater amino acid identity to GenBank Accession No. NP_{—}055694 or a fragment thereof and having at least one P2Y14 receptor biological activity. Human P2Y14 receptors are described, for example, by Lee et al., “P2Y-like receptor, GPR105 (P2Y₁₄), identifies and mediates chemotaxis of bone-marrow hematopoietic stem cells” GENES & DEVELOPMENT 17:1592-1604, 2003, which is hereby incorporated by reference in its entirety. Other exemplary P2Y14 receptor amino acid sequences include, for example, GenBank Accession No. Q 15391 and BAA05039. The P2Y14 receptor is also known in the art as GPR 105 and SC-GPR.

By “P2Y14 receptor biological activity” is meant a G protein receptor activity, such as calcium influx, uridine 5′-diphosphoglucose (UDP-glucose) binding, mediation of bone marrow hematopoietic cell chemotaxis, or other activity relating to hematopoietic stem cell growth, proliferation or survival.

By “P2Y14 receptor inhibitor” is meant any agent that reduces or eliminates the expression or biological activity of the P2Y14 receptor.

By “positioned for expression” is meant that the polynucleotide of the invention (e.g., a DNA molecule) is positioned adjacent to a DNA sequence that directs transcription and translation of the sequence.

By “progenitor cell” is meant a lineage-committed cell derived from a stem cell.

Progenitor cells may retain multipotency in their differentiation capacities, but have a significantly reduced self-renewal ability.

By “reference” is meant a standard or control condition.

The term “self renewal” as used herein refers to the process by which a stem cell divides to generate one (asymmetric division) or two (symmetric division) daughter cells with development potentials that are indistinguishable from those of the mother cell. Self-renewal involves both proliferation and the maintenance of an undifferentiated state.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

The term “stem cell” is meant a pluripotent cell having the capacity to self-renew and to differentiate into multiple cell lineages.

By “stem cell generation” is meant any biological process that gives rise to stem cells. Such processes include the proliferation of existing stem cells or stem cell self-renewal.

By “subject” is meant a mammal, including, but not limited to, a human or non-human mammal, such as a bovine, equine, canine, ovine, or feline.

By “syngeneic,” as used herein, refers to cells of a different subject that are genetically identical to the cell in comparison.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

The term “xenogeneic,” as used herein, refers to cells of a different species to the cell in comparison.

Other definitions appear in context throughout this disclosure.

### METHODS OF THE INVENTION

As described below, the present invention features methods and compositions for preserving stem cell function. In particular, the invention provides therapeutic or prophylactic methods that can increase survival, growth and/or proliferation of stem cells, and iurther, protect endogenous stem cells in settings of injury (e.g., chemotherapy). Such methods and compositions are useful for treating patients, such as transplant recipients or subjects having abnormal blood cell disorders (e.g., leukemia or myelodysplasia), that require an increase in the number of stem cells present in their bone marrow. The invention is based in part on the discovery that stem cells lacking the P2Y receptor-14 (P2Y14), tenned P2Y14^{-/-} stem cells, manifest a distinct advantage in repopulating irradiated host bone marrow.

Accordingly, the invention provides compositions and methods for reducing P2Y14 receptor polypeptide or polynucleotide expression in stem cells. Such compositions and methods are an improvement to existing techniques for repopulating bone marrow in patients in need thereof, for example, in blood and/or stem cell transplant patients whose bone marrow is depleted of hematopoietic stem cells following chemotherapy. In one embodiment, methods for modulating stem cells, which include a variety of stem cell types, are useful for enhancing stem cell expansion ex vivo or in vivo. The present invention is not limited to methods for enhancing hematopoietic stem cell expansion, but is broadly applicable to a variety of stem cells. Compositions and methods that inhibit P2Y14 expression or activity are useful for expanding stem cell populations in vivo and in vitro.

### P2Y14

Nucleotides and their conjugates serve as highly localized signal transmitters when located extracellularly and participate in stress or injury responses through P2 receptors that are either 7-membrane spanning (P2Y) or ATP-gated ion channels (P2X). The P2Y14 receptor has been shown to bind UTP-glucose, -galactose and -galactosamine and has a highly restricted expression that is limited to expression in adult human bone marrow to G₀CD34⁺CD38⁻-quiescent hematopoietic stem cells (HSCs). (GENES & DEVELOPMENT 17:1592-1604, 2003).

P2Y14 has a signature motif similar to a motif of the chemokine receptor family and with a nucleic acid sequence identical to the sequence of a previously identified gene, KIAA0001 (GenBank accession number D13626 or NM₁₃ 014879, SEQ ID No.: 10 of U.S. Ser. No. 10/433,146; the amino acid sequence is provided at SEQ ID NO:1 of U.S. Ser. No. 10/433,146). KIAA0001 was originally isolated from a cDNA library of human immature myeloid cell line KG-1 (Numura, N. et al. DNA Research, 1994, 1:47-56) and was characterized as a G-protein coupled receptor. It was not until recently that a function was assigned to this molecule (Chambers, J K et al., J. Biol. Chem., 2000, 275:1067-71).

### Stem Cells

Although the specific Examples described below relate to methods of enhancing hematopoietic stem cell growth, proliferation and survival by reducing P2Y14 receptor expression in the bone marrow, the invention is not so limited. The P2Y14 receptor is likely to function in regulating the size of a variety of stem cell pools. Stem cells of the present invention (e.g., embryonic stem cells, mesenchymal stem cells, hematopoietic stem cells) include all those known in the art that have been identified in mammalian organs or tissues. The best characterized is the hematopoietic stem cell. The hematopoietic stem cell, isolated from bone marrow, blood, cord blood, fetal liver and yolk sac, is the cell that generates blood cells or following transplantation reinitiates multiple hematopoietic lineages and can reinitiate hematopoiesis for the life of a recipient. (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827; Hill, B., et al. 1996.) When transplanted into lethally irradiated animals or humans, hematopoietic stem cells can repopulate the erythroid, neutrophil-macrophage, megakaryocyte and lymphoid hematopoietic cell pool. In vitro, hematopoietic stem cells can be induced to undergo at least some self-renewing cell divisions and can be induced to differentiate to the same lineages observed in vivo.

It is well known in the art that hematopoietic cells include pluripotent stem cells, multipotent progenitor cells (e.g., a lymphoid stem cell), and/or progenitor cells committed to specific hematopoietic lineages. The progenitor cells committed to specific hematopoietic lineages may be of T cell lineage, B cell lineage, dendritic cell lineage, Langerhans cell lineage and/or lymphoid tissue-specific macrophage cell lineage.

Hematopoietic stem cells can be obtained from blood products. A “blood product” as used in the present invention defines a product obtained from the body or an organ of the body containing cells of hematopoietic origin. Such sources include unfractionated bone marrow, umbilical cord, peripheral blood, liver, thymus, lymph and spleen. It will be apparent to those of ordinary skill in the art that all of the aforementioned crude or unfractionated blood products can be enriched for cells having “hematopoietic stem cell” characteristics in a number of ways. For example, the blood product can be depleted from the more differentiated progeny. The more mature, differentiated cells can be selected against, via cell surface molecules they express. Additionally, the blood product can be fractionated selecting for CD34⁺ cells. CD34⁺ cells are thought in the art to include a subpopulation of cells capable of self-renewal and pluripotentiality. Such selection can be accomplished using, for example, commercially available magnetic anti-CD34 beads (Dynal, Lake Success, N.Y.). Unfractionated blood products can be obtained directly from a donor or retrieved from cryopreservative storage.

In preferred embodiments of the invention, the hematopoietic stem cells may be harvested prior to treatment with a P2Y14 receptor inhibitor. “Harvesting” hematopoietic progenitor cells is defined as the dislodging or separation of cells from the matrix. This can be accomplished using a number of methods, such as enzymatic, non-enzymatic, centrifugal, electrical, or size-based methods, or preferably, by flushing the cells using media (e.g. media in which the cells are incubated). The cells can be further collected, separated, and further expanded generating even larger populations of differentiated progeny.

Methods for isolation of hematopoietic stem cells are well-known in the art, and typically involve subsequent purification techniques based on cell surface markers and functional characteristics. The hematopoietic stem and progenitor cells can be isolated from bone marrow, blood, cord blood, fetal liver and yolk sac, and give rise to multiple hematopoietic lineages and can reinitiate hematopoiesis for the life of a recipient. (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827; Hill, B., et al. 1996.) For example, for isolating hematopoietic stem and progenitor cells from peripheral blood, blood in PBS is loaded into a tube of Ficoll (Ficoll-Paque, Amersham) and centrifuged at 1500 rpm for 25-30 minutes. After centrifugation the white center ring is collected as containing hematopoietic stem cells.

Stem cells of the present invention also include embryonic stem cells. The embryonic stem (ES) cell has unlimited self-renewal and pluripotent differentiation potential (Thomson, J. et al. 1995; Thomson, J. A. et al. 1998; Shamblott, M. et al. 1998; Williams, R. L. et al. 1988; Orkin, S. 1998; Reubinoff, B. E., et al. 2000). These cells are derived from the inner cell mass (ICM) of the pre-implantation blastocyst (Thomson, J. et al. 1995; Thomson, J. A. et al. 1998; Martin, G. R. 1981), or can be derived from the primordial germ cells from a post-implantation embryo (embryonal germ cells or EG cells). ES and/or EG cells have been derived from multiple species, including mouse, rat, rabbit, sheep, goat, pig and more recently from human and human and non-human primates (U.S. Pat. Nos. 5,843,780 and 6,200,806).

Embryonic stem cells are well known in the art. For example, U.S. Pat. Nos. 6,200,806 and 5,843,780 refer to primate, including human, embryonic stem cells. U.S. Patent Applications Nos. 20010024825 and 20030008392 describe human embryonic stem cells. U.S. Patent Application No. 20030073234 describes a clonal human embryonic stem cell line. U.S. Pat. No. 6,090,625 and U.S. Patent Application No. 20030166272 describe an undifferentiated cell that is stated to be pluripotent. U.S. Patent Application No. 20020081724 describes what are stated to be embryonic stem cell derived cell cultures.

Stem cells of the present invention also include mesenchymal stem cells. Mesenchymal stem cells, or “MSCs” are well known in the art. MSCs, originally derived from the embryonal mesoderm and isolated from adult bone marrow, can differentiate to form muscle, bone, cartilage, fat, marrow stroma, and tendon. During embryogenesis, the mesoderm develops into limb-bud mesoderm, tissue that generates bone, cartilage, fat, skeletal muscle and endothelium. Mesoderm also differentiates to visceral mesoderm, which can give rise to cardiac muscle, smooth muscle, or blood islands consisting of endothelium and hematopoietic progenitor cells. Primitive mesodermal or MSCs, therefore, could provide a source for a number of cell and tissue types. A number of MSCs have been isolated. (See, for example, Caplan, A., et al., U.S. Pat. No. 5,486,359; Young, H., et al., U.S. Pat. No. 5,827,735; Caplan, A., et al., U.S. Pat. No. 5,811,094; Bruder, S., et al., U.S. Pat. No. 5,736,396; Caplan, A., et al., U.S. Pat. No. 5,837,539; Masinovsky, B., U.S. Pat. No. 5,837,670; Pittenger, M., U.S. Pat. No. 5,827,740; Jaiswal, N., et al., (1997). J. Cell Biochem. 64(2):295-312; Cassiede P., et al., (1996). J Bone Miner Res. 9:1264-73; Johnstone, B., et al., (1998) Exp Cell Res. 1:265-72; Yoo, et al., (1998) J Bon Joint Surg Am. 12:1745-57; Gronthos, S., et al., (1994). Blood 84:4164-73); Pittenger, et al., (1999). Science 284:143-147.

Mesenchymal stem cells are believed to migrate out of the bone marrow, to associate with specific tissues, where they will eventually differentiate into multiple lineages. Enhancing the growth and maintenance of mesenchymal stem cells, in vitro or ex vivo will provide expanded populations that can be used to generate new tissue, including breast, skin, muscle, endothelium, bone, respiratory, urogenital, gastrointestinal connective or fibroblastic tissues.

In certain embodiments, where a stem cell expresses P2Y14 receptor, the stem cell can be treated with a P2Y14 receptor inhibitor in vitro or ex vivo. Biological samples may comprise mixed populations of cells, which can be purified to a degree sufficient to produce a desired effect. Those skilled in the art can readily determine the percentage of stem cells or their progenitors in a population using various well-known methods, such as fluorescence activated cell sorting (FACS). Purity of the stem cells can be determined according to the genetic marker profile within a population. Dosages can be readily adjusted by those skilled in the art (e.g., a decrease in purity may require an increase in dosage).

In several embodiments, it will be desirable to first purify the cells. Stem cells of the invention preferably comprise a population of cells that have about 50-55%, 55-60%, 60-65% and 65-70% purity (e.g., non-stem and/or non-progenitor cells have been removed or are otherwise absent from the population). More preferably the purity is about 70-75%, 75-80%, 80-85%; and most preferably the purity is about 85-90%, 90-95%, and 95-100%. Purified populations of stem cells of the invention can be contacted with an P2Y14 receptor inhibitor before, after or concurrently with purification steps and administered to the subject.

### Stem Cell Culture

Once obtained from a desired source, contacting of a stem cell with an P2Y14 receptor inhibitor may, if desired, occur in culture. Employing the culture conditions described in greater detail below, it is possible to preserve stem cells of the invention and to stimulate the expansion of stem cell number and/or colony forming unit potential. In all of the in vitro and ex vivo culturing methods according to the invention, except as otherwise provided, the media used is that which is conventional for culturing cells. Appropriate culture media can be a chemically defined serum-free media such as the chemically defined media RPMI, DMEM, Iscove's, etc or so-called “complete media”. Typically, serum-free media are supplemented with human or animal plasma or serum. Such plasma or serum can contain small amounts of hematopoietic growth factors. The media used according to the present invention, however, can depart from that used conventionally in the prior art. Suitable chemically defined serum-free media are described in U.S. Ser. No. 08/464,599 and WO96/39487, and “complete media” are described in U.S. Pat. No. 5,486,359.

Treatment of the stem cells of the invention with P2Y14 receptor inhibitors may involve variable parameters depending on the particular type of inhibitor used. For example, ex vivo treatment of stem cells (e.g., bone marrow derived cells) with RNAi constructs that inhibit P2Y14 receptor expression may have a rapid effect (e.g., within 1-5 hours post transfection) while treatment with a chemical agent may require extended incubation periods (e.g., 24-48 hours). It is also possible to co-culture the stem cells treated according to the invention with additional agents that promote stem cell maintenance and expansion. It is well within the level of ordinary skill in the art for practitioners to vary the parameters accordingly.

The growth agents of particular interest in connection with the present invention are hematopoietic growth factors. By hematopoietic growth factors, it is meant factors that influence the survival or proliferation of hematopoietic stem cells. Growth agents that affect only survival and proliferation, but are not believed to promote differentiation, include the interleukins 3, 6 and 11, stem cell factor and FLT-3 ligand. The foregoing factors are well known to those of ordinary skill in the art and most are commercially available. They can be obtained by purification, by recombinant methodologies or can be derived or synthesized synthetically.

Thus, when cells are cultured without any of the foregoing agents, it is meant herein that the cells are cultured without the addition of such agent except as may be present in serum, ordinary nutritive media or within the blood product isolate, unfractionated or fractionated, which contains the hematopoietic stem and progenitor cells.

### Methods for Creating Genetically Altered Stem Cells

Genetic alteration of a stem cell includes all transient and stable changes of the cellular genetic material which are created by the addition of exogenous genetic material. In one embodiment, a population of cells that includes cells present in a stem cell niche is transfected with an P2Y14 receptor inhibitory nucleic acid molecule (e.g., siRNA, shRNA, antisense oligonucleotides). Such nucleic acid molecules inhibit the expression of P2Y14 receptor. In one approach, an inhibitory nucleic acid molecule is introduced directly into a target cell, such as a bone marrow derived cell, such that the inhibitory nucleic acid molecule reduces expression of P2Y14 receptor in the cell. In another approach, the target cell is transduced with an expression vector that encodes an inhibitory nucleic acid molecule. Expression of the P2Y14 receptor inhibitory nucleic acid molecule in the target cell reduces P2Y14 receptor expression. Other exemplary genetic alterations include any gene therapy procedure, such as introduction of a functional gene to replace a mutated or nonexpressed gene, introduction of a vector that encodes a dominant negative gene product, introduction of a vector engineered to express a ribozyme and introduction of a gene that encodes a therapeutic gene product. Natural genetic changes such as the spontaneous rearrangement of a T cell receptor gene without the introduction of any agents are not included in this embodiment. Exogenous genetic material includes nucleic acids or oligonucleotides, either natural or synthetic, that are introduced into the stem cells. The exogenous genetic material may be a copy of that which is naturally present in the cells, or it may not be naturally found in the cells. It typically is at least a portion of a naturally occurring gene which has been placed under operable control of a promoter in a vector construct.

Various techniques may be employed for introducing nucleic acids into cells. Such techniques include transfection of nucleic acid-CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid according to the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

One method of introducing exogenous genetic material into cells involves transducing the cells in situ on the matrix using replication-deficient retroviruses. Replication-deficient retroviruses are capable of directing synthesis of all virion proteins, but are incapable of making infectious particles. Accordingly, these genetically altered retroviral vectors have general utility for high-efficiency transduction of genes in cultured cells, and specific utility for use in the method of the present invention. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art.

Because viruses insert efficiently a single copy of the gene encoding the therapeutic agent into the host cell genome, retroviruses permit the exogenous genetic material to be passed on to the progeny of the cell when it divides. In addition, gene promoter sequences in the LTR region have been reported to enhance expression of an inserted coding sequence in a variety of cell types. However, using a retrovirus expression vector may result in (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. Despite these apparent limitations, delivery of a therapeutically effective amount of a therapeutic agent via a retrovirus can be efficacious if the efficiency of transduction is high and/or the number of target cells available for transduction is high.

Yet another viral candidate useful as an expression vector for transformation of cells is the adenovirus, a double-stranded DNA virus. Like the retrovirus, the adenovirus genome is adaptable for use as an expression vector for gene transduction, i.e., by removing the genetic information that controls production of the virus itself. Because the adenovirus functions usually in an extrachromosomal fashion, the recombinant adenovirus does not have the theoretical problem of insertional mutagenesis. On the other hand, adenoviral transformation of a target cell may not result in stable transduction. However, more recently it has been reported that certain adenoviral sequences confer intrachromosomal integration specificity to carrier sequences, and thus result in a stable transduction of the exogenous genetic material.

Thus, as will be apparent to one of ordinary skill in the art, a variety of suitable vectors are available for transferring exogenous genetic material into cells. The selection of an appropriate vector to deliver an agent and the optimization of the conditions for insertion of the selected expression vector into the cell, are within the scope of one of ordinary skill in the art without the need for undue experimentation. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any nontranslated DNA sequence which works contiguous with the coding sequence (in cis) to change the basal transcription level dictated by the promoter. Preferably, the exogenous genetic material is introduced into the cell genome immediately downstream from the promoter so that the promoter and coding sequence are operatively linked so as to permit transcription of the coding sequence. A preferred retroviral expression vector includes an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., 1991, Proc. Natl. Acad. Sci. USA, 88:4626-4630), adenosine deaminase, phosphoglycerol kinase (PGK), pyruvate kinase, phosphoglycerol mutase, the actin promoter (Lai et al., 1989, Proc. Natl. Acad. Sci. USA, 86:10006-10010), and other constitutive promoters known to those of skill in the art. In addition, many viral promoters function constitutively in eukaryotic cells. These include: the early and late promoters of SV40; the long terminal repeats (LTRS) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of an agent in the genetically modified cell. Selection and optimization of these factors for delivery of an is deemed to be within the scope of one of ordinary skill in the art without undue experimentation, taking into account the above-disclosed factors.

In addition to at least one promoter and at least one heterologous nucleic acid, the expression vector preferably includes a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence is deemed to be within the scope of one of ordinary skill in the art without undue experimentation.

### Methods of Using Inhibitory Nucleic Acids and Antibodies

The inhibitory nucleic acid molecules of the present invention may be employed as double-stranded RNAs for RNA interference (RNAi)-mediated knock-down of P2Y14 receptor expression. In one approach, P2Y14 receptor expression is reduced in a stem cell. In one exemplary approach, P2Y14 receptor expression is reduced in a hematopoietic stem cell. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, double-stranded RNA (dsRNA) molecule is made that includes between eight and twenty-five consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs consist of a stem-loop structure with optional 3′UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ U overhang in the expressed shRNA, which is similar to the 3′ overhangs of synthetic siRNAs. Additional methods for expressing the shRNA in mammalian cells are described in the references cited above.

Inhibitory nucleic acid molecules that target the P2Y14 receptor, such as siRNAs and shRNAs, for use in practicing the methods of the invention are commercially available, for example, from Santa Cruz Biotechnology Inc, OriGene Technologies and Sigma Aldrich. Inhibitory oligonucleotides are described, for example, in U.S. Patent Application Publication No. US2007/0092913, the contents of which are incorporated herein by reference.

Antibodies that block P2Y14 receptor binding have also been described, for example, by Lee B C et al. (2003) Genes Dev; 17:1592-604, the contents of which are incorporated herein by reference. Methods of raising antibodies against target receptors, such as the P2Y14 receptor, are well known in the art and can be performed as needed to generate antibodies for use according to the methods of the invention.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Treatment Methods Related to Stem Cell Expansion

In one aspect, the methods of the invention can be used to treat any disease or disorder in which it is desirable to increase the amount of stem cells and support the maintenance or survival of stem cells. Preferably, the stem cells are hematopoietic stem cells.

Frequently, subjects in need of the inventive treatment methods will be those undergoing or expecting to undergo an immune cell depleting treatment such as chemotherapy. Most chemotherapy agents used act by killing all cells going through cell division. Bone marrow is one of the most prolific tissues in the body and is therefore often the organ that is initially damaged by chemotherapy drugs. The result is that blood cell production is rapidly destroyed during chemotherapy treatment, and chemotherapy must be terminated to allow the hematopoietic system to replenish the blood cell supplies before a patient is re-treated with chemotherapy.

Thus, methods of the invention can be used, for example, to treat patients requiring a bone marrow transplant or a hematopoietic stem cell transplant, such as cancer patients undergoing chemo and/or radiation therapy. Methods of the present invention are particularly useful in the treatment of patients undergoing chemotherapy or radiation therapy for cancer, including patients suffering from myeloma, non-Hodgkin's lymphoma, Hodgkins lyphoma, or leukaemia.

Disorders treated by methods of the invention can be the result of an undesired side effect or complication of another primary treatment, such as radiation therapy, chemotherapy, or treatment with a bone marrow suppressive drug, such as zidovadine, chloramphenical or gangciclovir. Such disorders include neutropenias, anemias, thrombocytopenia, and immune dysfunction. In addition, methods of the invention can be used to treat damage to the bone marrow caused by unintentional exposure to toxic agents or radiation.

Methods of the invention can further be used as a means to increase the amount of mature cells derived from hematopoietic stem cells (e.g., erythrocytes). For example, disorders or diseases characterized by a lack of blood cells, or a defect in blood cells, can be treated by increasing the pool of hematopoietic stem cells. Such conditions include thrombocytopenia (platelet deficiency), and anemias such as aplastic anemia, sickle cell anemia, Fanconi's anemia, and acute lymphocytic anemia. In addition to the above, further conditions which can benefit from treatment using methods of the invention include, but are not limited to, lymphocytopenia, lymphorrhea, lymphostasis, erythrocytopenia, erthrodegenerative disorders, erythroblastopenia, leukoerythroblastosis; erythroclasis, thalassemia, myelofibrosis, thrombocytopenia, disseminated intravascular coagulation (DIC), immune (autoimmune) thrombocytopenic purpura (ITP), HIV inducted ITP, myelodysplasia; thrombocytotic disease, thrombocytosis, congenital neutropenias (such as Kostmann's syndrome and Schwachman-Diamond syndrome), neoplastic associated-neutropenias, childhood and adult cyclic neutropaenia; post-infective neutropaenia; myelo-dysplastic syndrome; and neutropaenia associated with chemotherapy and radiotherapy. The disorder to be treated can also be the result of an infection (e.g., viral infection, bacterial infection or fungal infection) causing damage to stem cells.

Immunodeficiencies, such as T and/or B lymphocytes deficiencies, or other immune disorders, such as rheumatoid arthritis and lupus, can also be treated according to the methods of the invention. Such immunodeficiencies may also be the result of an infection (for example infection with HIV leading to AIDS), or exposure to radiation, chemotherapy or toxins.

Also benefiting from treatment according to methods of the invention are individuals who are healthy, but who are at risk of being affected by any of the diseases or disorders described herein (“at-risk” individuals). At-risk individuals include, but are not limited to, individuals who have a greater likelihood than the general population of becoming cytopenic or immune deficient. Individuals at risk for becoming immune deficient include, but are not limited to, individuals at risk for HIV infection due to sexual activity with HIV-infected individuals; intravenous drug users; individuals who may have been exposed to HIV-infected blood, blood products, or other HIV-contaminated body fluids; babies who are being nursed by HIV-infected mothers; individuals who were previously treated for cancer, e.g., by chemotherapy or radiotherapy, and who are being monitored for recurrence of the cancer for which they were previously treated; and individuals who have undergone bone marrow transplantation or any other organ transplantation, or patients anticipated to undergo chemotherapy or radiation therapy or be a donor of stem cells for transplantation.

A reduced level of immune function compared to a normal subject can result from a variety of disorders, diseases infections or conditions, including immunosuppressed conditions due to leukemia, renal failure; autoimmune disorders, including, but not limited to, systemic lupus erythematosus, rheumatoid arthritis, auto-immune thyroiditis, scleroderma, inflammatory bowel disease; various cancers and tumors; viral infections, including, but not limited to, human immunodeficiency virus (HIV); bacterial infections; and parasitic infections.

A reduced level of immune function compared to a normal subject can also result from an immunodeficiency disease or disorder of genetic origin, or due to aging. Examples of these are immunodeficiency diseases associated with aging and those of genetic origin, including, but not limited to, hyperimmunoglobulin M syndrome, CD40 ligand deficiency, IL-2 receptor deficiency, γ-chain deficiency, common variable immunodeficiency, Chediak-Higashi syndrome, and Wiskott-Aldrich syndrome.

A reduced level of immune function compared to a normal subject can also result from treatment with specific pharmacological agents, including, but not limited to chemotherapeutic agents to treat cancer; certain immunotherapeutic agents; radiation therapy; immunosuppressive agents used in conjunction with bone marrow transplantation; and immunosuppressive agents used in conjunction with organ transplantation.

Where the stem cells to be provided to a subject in need of such treatment are hematopoietic stem cells, they are most commonly obtained from the bone marrow of the subject or a compatible donor. Bone marrow cells can be easily isolated using methods know in the art. For example, bone marrow stem cells can be isolated by bone marrow aspiration. U.S. Pat. No. 4,481,946, incorporated herein expressly by reference, describes a bone marrow aspiration method and apparatus, wherein efficient recovery of bone marrow from a donor can be achieved by inserting a pair of aspiration needles at the intended site of removal. Through connection with a pair of syringes, the pressure can be regulated to selectively remove bone marrow and sinusoidal blood through one of the aspiration needles, while positively forcing an intravenous solution through the other of the aspiration needles to replace the bone marrow removed from the site. The bone marrow and sinusoidal blood can be drawn into a chamber for mixing with another intravenous solution and thereafter forced into a collection bag. The heterogeneous cell population can be further purified by identification of cell-surface markers to obtain the bone marrow derived germline stem cell compositions for administration into the reproductive organ of interest.

U.S. Pat. No. 4,486,188 describes methods of bone marrow aspiration and an apparatus in which a series of lines are directed from a chamber section to a source of intravenous solution, an aspiration needle, a second source of intravenous solution and a suitable separating or collection source. The chamber section is capable of simultaneously applying negative pressure to the solution lines leading from the intravenous solution sources in order to prime the lines and to purge them of any air. The solution lines are then closed and a positive pressure applied to redirect the intravenous solution into the donor while negative pressure is applied to withdraw the bone marrow material into a chamber for admixture with the intravenous solution, following which a positive pressure is applied to transfer the mixture of the intravenous solution and bone marrow material into the separating or collection source.

It will be apparent to those of ordinary skill in the art that the crude or unfractionated bone marrow can be enriched for cells having desired “stem cell” characteristics. Some of the ways to enrich include, e.g., depleting the bone marrow from the more differentiated progeny.

The more mature, differentiated cells can be selected against, via cell surface molecules they express. Enriched bone marrow immunophenotypic subpopulations include but are not limited to populations sorted according to their surface expression of Lin, cKit and Sca-1 (e.g., LK+S+ (Lin-cKit⁺Sca1⁺), LK-S+ (Lin-cKit⁺Sca1⁺), and LK+S− (Lin-cKit⁺Sca1⁺)).

Bone marrow can be harvested during the lifetime of the subject. However, harvest prior to illness (e.g., cancer) is desirable, and harvest prior to treatment by cytotoxic means (e.g., radiation or chemotherapy) will improve yield and is therefore also desirable.

Accordingly, the present invention provides methods of treating disease and/or disorders or symptoms thereof which comprise administering a therapeutically effective amount of a pharmaceutical composition comprising a stem cell treated as described herein to a subject (e.g., a mammal, such as a human). Thus, one embodiment is a method of treating a subject having a disease characterized by a lack of blood cells. The method includes the step of administering to the mammal a therapeutic amount of a stem cell (e.g., hematopoietic stem cell), or mixture comprising such cell types treated with a P2Y14 receptor inhibitor as described herein sufficient to treat a disease or disorder or symptom thereof, under conditions such that the disease or disorder is treated.

The methods herein include administering to the subject (including a subject identified as in need of such treatment) an effective amount of a stem cell treated with an P2Y14 receptor inhibitor described herein, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of a pharmaceutical composition comprising a stem cell treated with a P2Y14 receptor inhibitor herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which a lack of blood cells may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with having a reduced number of stem cells, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy.

In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Administration of Stem Cells

Following treatment with a suitable P2Y14 receptor inhibitor, stem cells are administered according to methods known in the art. Such compositions may be administered by any conventional route, including injection or by gradual infusion over time. The administration may, depending on the composition being administered, for example, be, pulmonary, intravenous, intraperitoneal, intramuscular, intracavity, subcutaneous, or transdermal. The stem cells are administered in “effective amounts”, or the amounts that either alone or together with further doses produces the desired therapeutic response.

Administered cells of the invention can be autologous (“self”) or non-autologous (“non-self,” e.g., allogeneic, syngeneic or xenogeneic). Generally, administration of the cells can occur within a short period of time following P2Y14 receptor inhibitor treatment (e.g. 1, 2, 5, 10, 24 or 48 hours after treatment) and according to the requirements of each desired treatment regimen.

For example, where radiation or chemotherapy is conducted prior to administration, treatment, and transplantation of stem cells of the invention should optimally be provided within about one month of the cessation of therapy. However, transplantation at later points after treatment has ceased can be done with derivable clinical outcomes.

### Stem Cell Related Pharmaceutical Compositions

Following harvest and treatment with a suitable P2Y14 receptor inhibitor, stem cells may be combined with pharmaceutical excipients known in the art to enhance preservation and maintenance of the cells prior to administration. In some embodiments, cell compositions of the invention can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

A method to potentially increase cell survival when introducing the cells into a subject in need thereof is to incorporate stem cells of interest into a biopolymer or synthetic polymer. Depending on the subject's condition, the site of injection might prove inhospitable for cell seeding and growth because of scarring or other impediments. Examples of biopolymer include, but are not limited to, cells mixed with fibronectin, fibrin, fibrinogen, thrombin, collagen, and proteoglycans. This could be constructed with or without included expansion or differentiation factors. Additionally, these could be in suspension, but residence time at sites subjected to flow would be nominal. Another alternative is a three-dimensional gel with cells entrapped within the interstices of the cell biopolymer admixture. Again, expansion or differentiation factors could be included with the cells. These could be deployed by injection via various routes described herein.

Those skilled in the art will recognize that the components of the compositions should be selected to be chemically inert and will not affect the viability or efficacy of the stem cells or their progenitors as described in the present invention. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of stem cells is the quantity of cells necessary to achieve an optimal effect. Different scenarios may require optimization of the amount of cells injected into a tissue of interest. Thus, the quantity of cells to be administered will vary for the subject being treated. The precise determination of what would be considered an effective dose may be based on factors individual to each patient, including their size, age, sex, weight, and condition of the particular patient. As few as 100-1000 cells can be administered for certain desired applications among selected patients. Therefore, dosages can be readily ascertained by those skilled in the art from this disclosure and the knowledge in the art.

The skilled artisan can readily determine the amount of cells and optional additives, vehicles, and/or carrier in compositions and to be administered in methods of the invention. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Pharmaceutical Compositions

The invention provides a simple means for identifying compositions (including nucleic acids, peptides, small molecule inhibitors, and mimetics) capable of acting as therapeutics or as prophylactics to protect stem cell populations at risk of undergoing cell death, for example, to protect stem cells in patients undergoing chemotherapy. In particular, the invention provides inhibitors of a P2Y14 receptor that are useful for protecting a hematopoietic stem cell in a patient undergoing chemotherapy or for enhancing stem cell growth, proliferation or survival following stem cell transplantation.

Antagonists of the P2Y family include, but are not limited to Suramin (Yitzhaki et al., Biochem Pharmacol. 69(8):1215-23; 2005; Lambrecht et al., Curr Pharm Des. 8(26): 2371-99, 2002; and von Kugelgen, Pharmacol Ther. 110(3): 415-32, 2006), Pyridoxal-5′-phosphate-6-azophenyl-2,4-disulfonate (PPADS) (Yitzhaki et al., Biochem Pharmacol. 69(8):1215-23; 2005; Lambrecht et al., Curr Pharm Des. 8(26): 2371-99, 2002), Reactive blue (RB-2) (Yitzhaki et al., Biochem Pharmacol. 69(8): 1215-23; 2005; von Kugelgen, Pharmacol Ther. 110(3): 415-32, 2006), Montelukast (Mamedova Biochem Pharmacol. 71(1-2): 115-25, 2005), and Pranlukast (Mamedova Biochem Pharmacol. 71(1-2): 115-25, 2005).

Accordingly, a chemical entity discovered to have medicinal value using the methods described herein is useful as a drug or as information for structural modification of existing compounds, e.g., by rational drug design. For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplastic disease.

### Screening Assays

Screening methods of the invention can involve the identification f an P2Y14 receptor inhibitor that promotes survival or expansion of a population of stem cells. Such methods will typically involve contacting a population of cells that include stem cells and cells that express P2Y14 receptor with a suspected inhibitor in culture and quantitating the number of long-term repopulating cells produced as a result. A quantitative in vivo assay (for the determination of the relative frequency of long-term repopulating stem cells) based on competitive repopulation combined with limiting dilution analysis has been previously described in Schneider, T. E., et al. (2003) PNAS 100(20):11412-11417. Similarly, Zhang, J., et al. (2005 Gene Therapy 12:1444-1452) describes the injection of NOD/SCID mice with siRNA-treated lentiviral-transduced human CD34+ cells, followed by the killing of the mice and harvesting of the bone marrow mononuclear cells. The cells were subsequently stained with anti-human leukocyte marker antibodies for FACS analysis allowing the detection of the markers (and, thus, quantitation of the cells of interest). Comparison to an untreated control can be concurrently assessed. Where an increase in the number of long-term repopulating cells is detected relative to the control, the suspected inhibitor is determined to have the desired activity.

PCT application WO99/57245 (SmithKline Beecham Corporation) discloses methods of screening for agonists and antagonists of the interaction between the human KIAA0001 receptor and ligands thereof. As mentioned above, the human KIAA0001 receptor has the same sequence as the human P2Y14 receptor. One of ordinary skill in the art can use the human P2Y14 receptor antagonists using the methods described in PCT application WO99/57245

Human P2Y14 receptor antagonists, blocking agents or other binding agents which prevent P2Y14 receptor activity can be identified as previously described and then tested for their effects on hematopoietic stem cell function using any of the assays described herein or otherwise known in the art. For instance, in vitro and in vivo assays for enhancing mobilization of hematopoietic stem cells, in assays for calcium influx in response to UDP-glucose treatment, or in assays of cell survival, growth or proliferation. Such assays are known to the skilled artisan.

In practicing the screening methods of the invention, it may be desirable to employ a cell population that includes stem cells. In one embodiment, a purified population of stem cells is used. In other methods, the test agent is assayed using a biological sample rather than a purified population of stem cells. The term “biological sample” includes tissues, cells and biological fluids isolated from a subject, as well as tissues, cells and fluids present within a subject. Preferred biological samples include bone marrow and peripheral blood.

Increased amounts of long-term repopulating cells can be detected by an increase in gene expression of certain markers including, but not limited to, Hes-1, Bmi-1, Gfi-1, SLAM genes, CD51, GATA-2, Scl, P2y14, and CD34. These cells may also be characterized by a decreased or low expression of genes associated with differentiation. The level of expression of genes of interest can be measured in a number of ways, including, but not limited to: measuring the mRNA encoded by the genes; measuring the amount of protein encoded by the genes; or measuring the activity of the protein encoded by the genes.

The level of mRNA corresponding to a gene of interest can be determined both by in situ and by in vitro formats. The isolated mRNA can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe is sufficient to specifically hybridize under stringent conditions to mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the genes of interest described herein.

The level of mRNA in a sample can be evaluated with nucleic acid amplification, e.g., by reverse transcription-polymerase chain reaction (rtPCR) (Mullis (1987) U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the gene of interest being analyzed.

### Test Compounds and Extracts

In general, compounds capable of decreasing the expression or activity of an P2Y14 receptor polypeptide are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to decrease the expression or activity of an P2Y14 receptor polypeptide, further fractionation of the positive lead extract is necessary to isolate chemical constituents responsible for the observed effect. Thus, the goal of the extraction, fractionation, and purification process is the careful characterization and identification of a chemical entity within the crude extract that decreases the expression or activity of an P2Y14 receptor polypeptide. Methods of fractionation and purification of such heterogenous extracts are known in the art. If desired, compounds shown to be useful as therapeutics for supporting stem cell expansion are chemically modified according to methods known in the art.

### Kits

The invention provides kits for promoting stem cell survival, growth, or proliferation, of a stem cell into a tissue of a subject. In one embodiment, the kit includes a therapeutic composition containing an effective amount of an P2Y14 receptor inhibitor in unit dosage form. In one example, an effective amount of P2Y14 receptor is an amount sufficient to promote stem cell survival or self-renewal in a culture comprising a mixture of cell types that includes stem cells. In other embodiments, the kit comprises a sterile container that contains a therapeutic or prophylactic vaccine; such containers can be boxes, ampoules, bottles; vials, tubes, bags, pouches, blister-packs, or other suitable container forms known in the art. Such containers can be made of plastic, glass, laminated paper, metal foil, or other materials suitable for holding medicaments.

If desired an P2Y14 receptor inhibitor is provided together with instructions for administering it to a stem cell culture or to a tissue of a subject. The instructions will generally include information about the use of the composition for the expansion of a stem cell population or for the growth, proliferation or survival of a stem cell population in a tissue. In other embodiments, the instructions include at least one of the following: description of the P2Y14 receptor inhibitor; dosage schedule and administration for the expansion of a stem cell population, precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

In another aspect, the invention provides kits that feature an P2Y14 receptor polypeptide or nucleic acid molecule. Such compositions are generally useful for protecting a stem cell population that is at risk of undergoing cell death (e.g., a hematopoietic stem cell population in a patient having chemotherapy) or for enhancing stem cell transplantation (e.g., by increasing the survival, growth, or proliferation of a transplanted stem cell).

### Therapies for Enhancing Stem Cell Function

Agents that enhance stem cell function include those that increase the survival, growth, and proliferation of a stem cell. Such agents are useful in the methods of the invention. Methods of assaying cell growth and proliferation are known in the art. See, for example, Kittler et al. (Nature. 432 (7020):1036-40, 2004) and Miyamoto et al. (Nature 416(6883):865-9, 2002). Assays for cell proliferation generally involve the measurement of DNA synthesis during cell replication. In one embodiment, DNA synthesis is detected using labeled DNA precursors, such as ([³H]-Thymidine or 5-bromo-2*-deoxyuridine [BrdU], which are added to cells (or animals) and then the incorporation of these precursors into genomic DNA during the S phase of the cell cycle (replication) is detected (Ruefli-Brasse et al., Science 302(5650):1581-4, 2003; Gu et al., Science 302 (5644):445-9, 2003).

Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

Candidate agents that reduce stem cell death (e.g., decrease apoptosis) are also useful as therapeutics in the methods of the invention. Assays for measuring cell apoptosis are known to the skilled artisan. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

If desired, candidate agents selected using any of the screening methods described herein are tested for their efficacy using animal models. In one embodiment, mice are treated with chemotherapeutics human cells to reduce the population of hematopoietic stem cells. The mice are then administered (e.g., intraperitoneally) with vehicle (PBS) or a candidate compound (e.g., an inhibitor of a P2Y14 receptor) daily for a period of time to be empirically determined. Mice are then euthanized and tissues comprising hematopoietic stem cells (e.g., bone marrow) are collected and analyzed using methods described herein. Compositions that reduce hematopoietic stem cell death relative to control levels are expected to be efficacious as therapeutics or prophylactics for the treatment of the adverse consequences of chemotherapy in a subject (e.g., a human patient).

### Combination Therapies

Optionally, a therapeutic of the invention may be administered in combination with any other standard therapy for enhancing stem cell survival. Such therapies include the administration of factors that promote stem cell self-renewal, survival, or generation.

P2Y14 receptor inhibitors may be administered in combination with any other standard neoplasia therapy; such methods are known to the skilled artisan (e.g., Wadler et al., Cancer Res. 50:3473-86, 1990), and include, but are not limited to, chemotherapy, hormone therapy, immunotherapy, radiotherapy, and any other therapeutic method used for the treatment of neoplasia.

The present invention is additionally described by way of the following illustrative, non-limiting Examples that provide a better understanding of the present invention and of its many advantages.

### EXAMPLES

### Example 1

### P2Y14 Null Mice are Developmentally Normal

The definitive function of the P2Y14 receptor in hematopoietic stem cell regulation is now reported. This function was characterized using a P2Y14 null mouse. P2Y14^{-/-} mice backcrossed to C57B1/6 developed normally and have similar birth rates and body weights compared to their wild-type littermates. No differences were found in peripheral blood counts, marrow cellularity or stem cell enriched lineage-cKit⁺Sca1⁺ (LKS) and LKS CD34⁻ cells at nine weeks of age between P2Y14^{-/-}, +/− and +/+ littermates. Similarly, cell cycle status, in vitro colony-forming cell (CFC) capacity, 6-hour in vivo homing and in vivo colony-forming unit-spleen (CFU-S) function were all similar.

### Example 2

### P2Y^{-/-} Stem Cells Exhibited Enhanced Bone Marrow Engraftment

When stem cells were exposed to conditions of stress in irradiated host bone marrow, P2Y^{-/-} stem cells outperformed wild-type controls. Competitive repopulation assays compared engraftment of CD45.2⁺ P2Y14^{-/-} or +/+ littermate bone marrow at a 1:1 cell ratio with competitor male CD45.1+ B6.5JL into irradiated female CD45.1+ B6.5JL mice. P2Y^{-/-} stem cells manifested a distinct engraftment advantage at eighteen weeks (% CD45.2: 49.82% vs 27.10%; n=8 per group; p=0.0079, Mann-Whitney). Differentiation into all mature lineages was preserved. Quantitative, limit dilution competitive repopulation into irradiated recipients revealed a greater number of functional stem cells in the P2Y14^{-/-} bone marrow (1/23,120 vs. 1/63,876 Poisson; p=0.0359). Further, secondary transplantation revealed a repopulation advantage of P₂Y14^{-/-} cells at eighteen weeks (41.55% vs. 22.54%; n=16 per group; p=0.0022). These findings indicate that P2Y14 regulated stem cell number and function under conditions of stress in the bone marrow, reflecting a role for extracellular nucleotides in modulating the stem cell pool.

### Example 3

### P2Y₁₄^{-/-} HSCs, Unable to Detect UDP-glucose, Respond to Highly Proliferative Environments

Since nucleotide receptors have been shown to be important in response to inflammatory injury, the mechanisms which underlie the role of P2Y₁₄ were examined in models of bone marrow (BM) injury.

First, the cyclophosphamide (CTX) injury model was employed, where the mice were injected with CTX and BM was harvested at day 4 after injection for examination. In the CIX injury model, either sterile PBS or CTX at final concentration of 200 mg/kg mouse weight was injected intraperitoneally. At day 4 after injection, mice were euthanized and BM was obtained from femurs and tibiae of each mouse (n=5 per group).

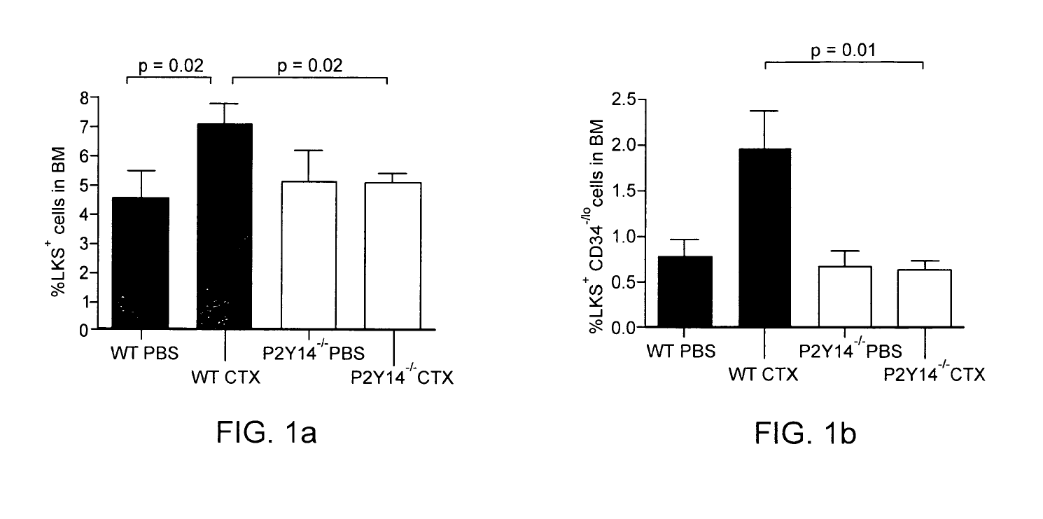

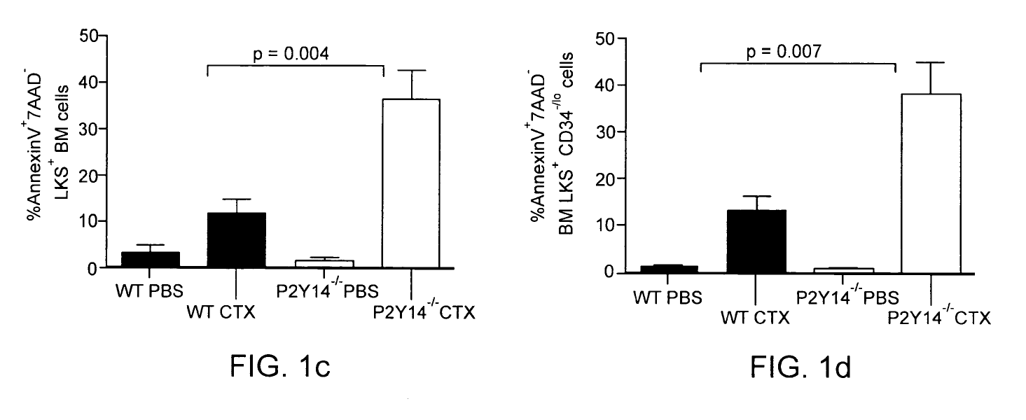

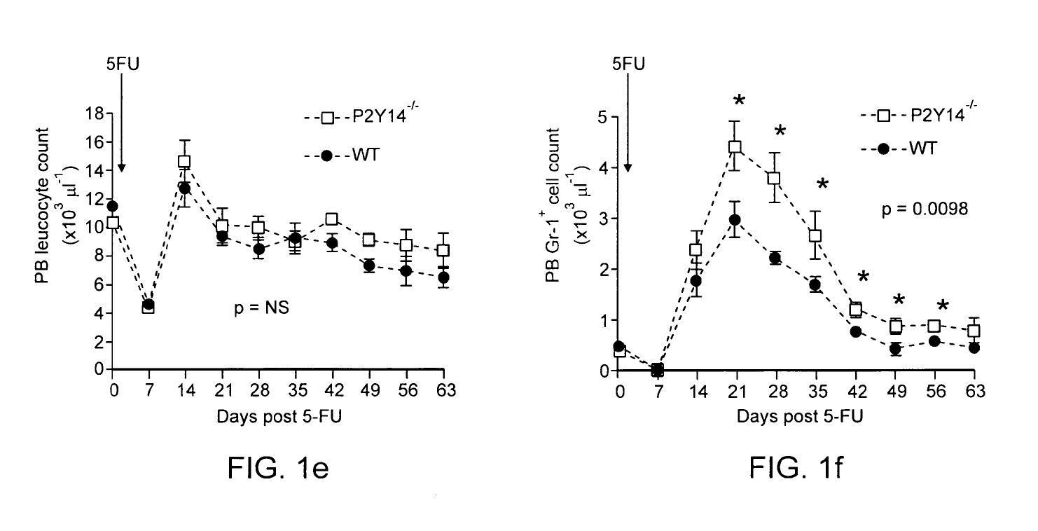

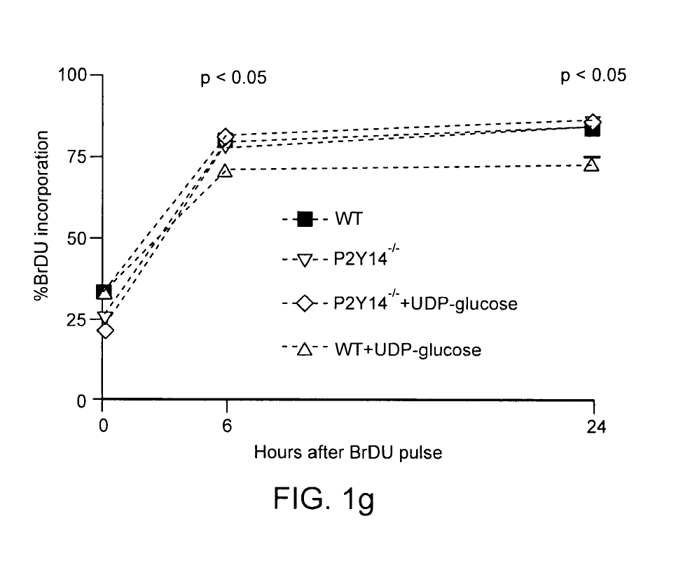

Using this model, it was determined that there is enrichment of immature HSC fraction of the BM in wildtype mice as expected, since CTX preferentially eliminates actively dividing cells and quiescent HSCs are relatively protected from the effects of CTX (FIG. 1a and 1b). In contrast, there is no such protection from CTX in P2Y₁₄^{-/-} BM, resulting in loss of enrichment for immature HSC fraction (FIG. 1a and 1b). This effect appears to be mediated through increased apoptosis in the immature HSC fraction exposed to CTX (FIGS. 1c and 1d).

Similar results were obtained when mice were subjected to BM injury using 5-fluorouracil (5FU) (data not shown). In the 5FU injury model, 150 mg/kg mouse weight 5FU was injected intraperitoneally at day 0 PB was obtained through tail vein bleeding for analysis weekly starting day 0 (n=10 per group). These results indicate that the presence of P2Y₁₄ provides protection from apoptosis.

Next, we examined the role of P2Y₁₄ in long-term hematopoietic recovery from BM injury. The mice were subjected to a single injection of 5FU and followed the peripheral blood (PB) leucocyte counts as well as PB granulocyte, B cell and T cell content by flow cytometry.

The staining and flow cytometric analysis of BM for LKS+ and CD34^{lo/-}LKS⁺ cells and of PB for B cell, T cell and granulocyte subsets has been previously described (Yuan, Y. et al. Nat. Cell Biol. 6, 436-442 (2004); Walkley, C. R. et al. Nat. Cell Biol. 7, 172-178 (2005)). Apoptosis was measured by AnnexinV and 7AAD staining as previously described Cheng, T. et al. Nat. Med. 6, 1235-1240 (2000).

When total PB leucocyte count was examined, there was an expected kinetic after 5FU injection for both ^{-/-} and ^{+/+} mice, that is, there was a rapid decline of leucocyte count leading to a nadir at day 7, followed by a rebound leucocytosis peaking at day 14 and gradual return to baseline leucocyte count at 3-4 weeks post-injection (FIG. 1e). Similarly, the examination of PB Gr-1⁺ granulocyte counts revealed that 5FU treatment of P2Y₁₄^{-/-} mice leads to an expected decline and nadir of granulocyte counts.

However, following the nadir, there is more rapid rebound of Gr-1⁺ cells in the PB of P2Y₁₄^{-/-} mice leading to an overcompensated Gr-1⁺ cell recovery that persists up to 9 weeks after injection (FIG. 1f). This suggests that there is an accentuated myelopoiesis in P2Y₁₄^{-/-} BM following chemical injury. These effects appear specific to chemical injury, since sublethal irradiation, known to damage both BM HSCs and stromal elements, at 2.5 and 4.0 Gy resulted in similar percentage and number of immature HSCs at days 4 and 7 post-irradiation.

A possible mechanism for these differences between the responses of P2Y₁₄^{-/-} and ^{+/+} BM to chemical injury is through induction or maintenance of HSC quiescence through P2Y₁₄ binding to its specific ligand, UDP-glucose. To test this hypothesis, the effect of UDP-glucose on BM HSCs that are put into cell cycle-promoting culture conditions in vitro was examined. When sorted BM LKS⁺ cells are put into culture containing cytokines, they undergo at least one division during a 24-hour period. After 3 days, the cells were exposed to UDP-glucose for 16 hours, followed by BrDU incorporation. 1×10⁵ BM LKS⁺ cells were obtained from 3 mice of each genotype by cell sorting and cultured in X-VIVO10 medium (Stem Cell Technologies, Vancouver) supplemented with 10 ng/ml SCF, 50 ng/ml TPO, 10 ng/ml Flt3L and 100 ng/ml IL3. After 3 days, the media was replaced with fresh X-VIVO10 medium supplemented with same cytokines without IL3, and 100 mM UDP-glucose or PBS were added to each well. After 16 hours, cells in each well were washed and pulsed for 45 minutes with BrDU (BD BioScience, San Jose). 1×10⁵ cells were harvested from each well at 6 and 24 hours and BrDU incorporation was measured by flow cytometry as per FITC BrDU Flow Kit instructions (BD BioScience, San Jose). All groups were in triplicate.

As expected, both P2Y₁₄^{-/-} and ^{+/+}LKS⁺ cells exhibit high levels of BrDU incorporation when stimulated with cytolines promoting cell cycling (FIG. 1g). Similarly, when cycling P2Y₁₄^{-/-} LKS⁺ cells are exposed to UDP-glucose, there is no change in BrDU incorporation. However, when cycling P2Y₁₄^{+/+} LKS⁺ cells are exposed to UDP-glucose, there is a significant slowing of cell cycling, as seen by lower levels of BrDU incorporation at both 6 and 24 hours after the BrDU pulse (FIG. 1g). From these results, it can be determined that the P2Y₁₄ protection from apoptosis following chemical injury is mediated through maintenance of relative quiescence.

The above models of BM injury result in damage to both BM hematopoietic cells as well as BM environment. The effect of injured BM environment to uninjured HSCs by HSC transplantation assays was examined. The transplantation assays follow previously described protocols (Cheng, T. et al. Nat. Med. 6, 1235-1240 (2000); Cheng, T. et al. Science 289, 1804-1808 (2000)). Briefly, female CD45.1⁺C57SJL recipients were lethally irradiated using 10 Gy irradiation in split doses. For 1:1 competitive serial transplantation, 2×10⁵ whole BM mononuclear cells obtained from each genotype (CD45.2⁺) and from male CD45.1⁺ C57SJL competitors were mixed and injected intravenously into recipients (n=10 per group). Engraftment was followed every 3 weeks starting week 6 following transplantation by PB flow cytometry for CD45.1 and 2 and B cell, T cell and granulocyte and monocyte lineages as previously described (Calvi, L. M. et al. Nature 425, 841-846 (2003)). At 18 weeks, the mice were euthanized and 2×10⁵ BM mononuclear cells from each mouse were transplanted into female CD45.1⁺ C57SJL secondary recipients (n=10 per group). Tertiary transplantation was performed in a similar manner. Statistical significance was determined using the Wilcoxon test. For limiting dilution serial transplantation, P2Y₁₄^{-/-} or ^{+/+} whole BM mononuclear cells were mixed with male CD45.1⁺C57SJL whole BM mononuclear cells at 1:1, 1:2 and 1:4 ratios and injected into lethally irradiated female CD45.1⁺ C57SJL mice. Engraftment, as defined as both % CD45.2⁺ PB mononuclear cells and % CD45.2⁺ PB Gr-1⁺ granulocytes>2.5%, was assessed at 18 weeks. CRU equivalents were calculated using L-Calc software (Stem Cell Technologies, Vancouver).

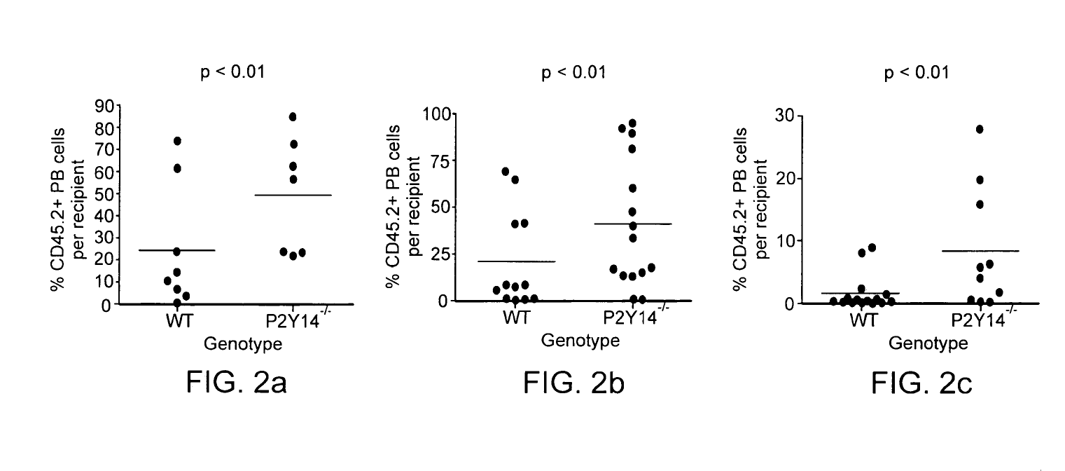

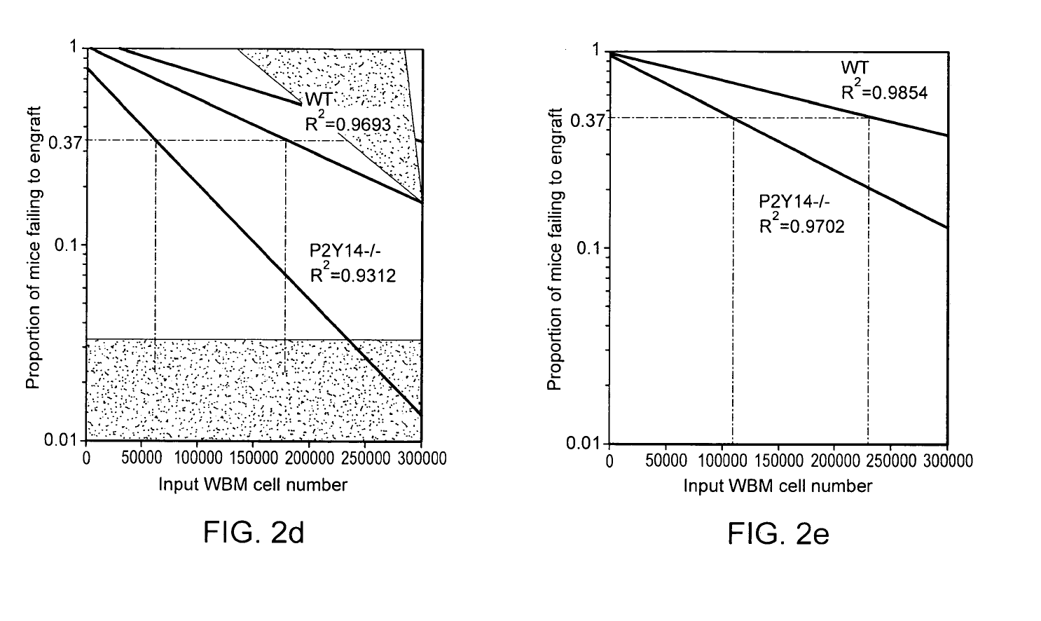

When P2Y₁₄^{-/-} and ^{+/+} whole BM cells were compared in competitive transplantation, P2Y₁₄^{-/-} whole BM resulted in superior engraftment of lethally irradiated primary recipients as measured by percentage of peripheral blood CD45.2⁺ leucocytes (FIG. 2a). In addition, P2Y₁₄^{-/-} whole BM HSCs performed better during serial transplantation, indicating superior self-renewal capacity of P2Y₁₄^{-/-} HSCs (FIGS. 2b and 2c). Limiting dilution competitive transplantation showed that P2Y₁₄^{-/-} whole BM contains equivalent of approximately three times greater engraftment capacity compared to ^{+/+} whole BM in lethally irradiated primary recipients (Table 1, FIG. 2d). This effect persists but is attenuated in secondary recipients (Table 1, FIG. 2e). From these results, it can be concluded that P2Y₁₄^{-/-} HSCs, when introduced into injured BM environment, has a greater engraftment and self-renewal capacity compared with ^{+/+} HSC.

TABLE
Quantification of greater engraftment capacity in P2Y₁₄^{-/-} whole BM.

| | WT | P2Y₁₄^{-/-} | p-value |
|---|---|---|---|
| Primary transplantation | | | |
| CRU equivalent | 1: 18 × 10⁴ | 1: 6.5 × 10⁴ | 0.0072 |
| Relative engraftment capacity | 1 | 2.77 | |
| Secondary transplantation | | | |
| CRU equivalent | 1: 2.8 × 10⁵ | 1: 1.3 × 10⁵ | 0.03 |
| Relative engraftment capacity | 0.64 | 1.38 | |

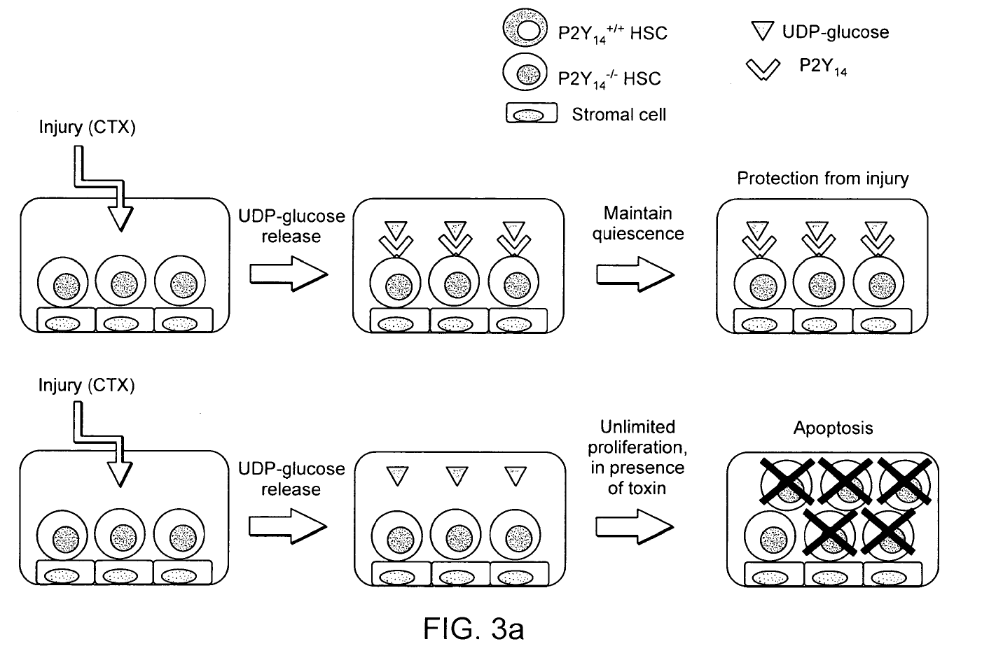

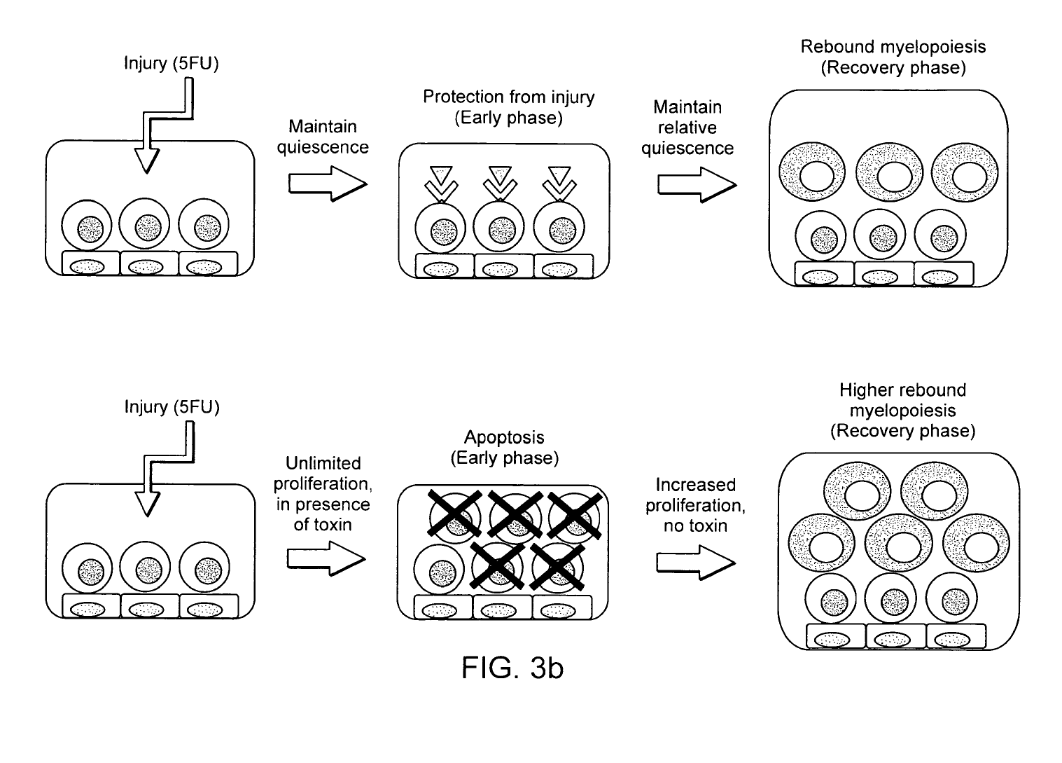

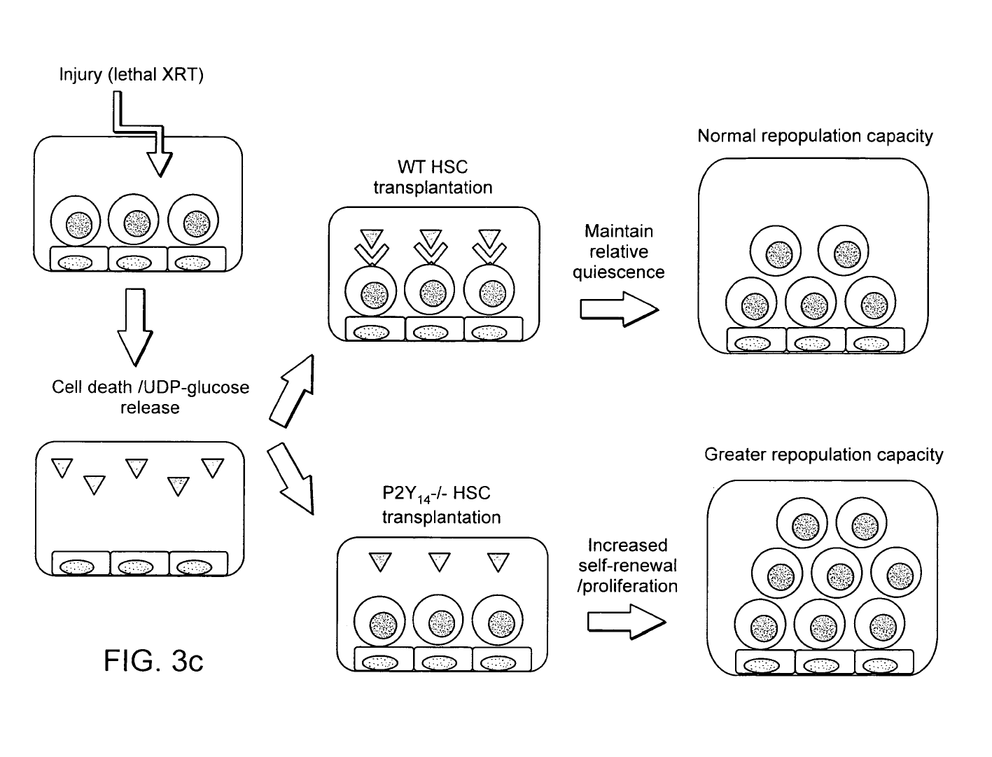

From these findings, the following model for the role of P2Y₁₄ in BM injury was developed (FIG. 3). During acute chemical injury of the BM, where toxic agent damaging both the BM HSCs and BM stromal elements are present, P2Y₁₄-expressing HSCs sense the “danger signal” through binding to UDP-glucose resulting in maintenance of relative quiescence leading to relative resistance to toxin-induced apoptosis (FIG. 3a). This is followed by a recovery phase, where the offending toxic agent is no longer present in the BM. Importantly, during this period, the lack of P2Y₁₄ results in more exuberant, overcompensated rebound myelopoiesis, since P2Y₁₄^{-/-} cells respond more readily to proliferative stimuli created by loss of differentiated progeny following chemical injury (FIG. 3b). In the setting of injury, newly introduced P2Y₁₄^{+/+} HSCs sense the presence of UDP-glucose and maintain relative quiescence while P2Y₁₄^{-/-} HSCs, unable to detect UDP-glucose, respond to highly a proliferative environment resulting from lethal irradiation and loss of both native HSCs and their differentiated progeny, by greater differentiation/proliferation and self-renewal, as shown by superior performance in transplantation assays (FIG. 3c).

Accordingly, the invention provides inhibitors of the P2Y 14 receptor (also known as GPR 105 and SC-GPR) to hematopoetic stem cells expressing the P2Y14 receptor to improve stem cell expansion and overall blood cell recovery in transplant recipients or subjects having abnormal blood cell disorders (e.g., leukemia or myelodysplasia). Exemplary inhibitors are siRNAs against the P2Y14 receptor. Small molecule inhibitors are contemplated.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for increasing stem cell expansion, the method comprising contacting a stem cell with an effective amount of an agent that inhibits P2Y14 receptor expression or biological activity, thereby increasing stem cell expansion.

2. The method of claim 1, wherein the stem cell is contacted in vivo or ex vivo.

3. The method of claim 1, wherein the stem cell is contacted in vivo in a subject having received a stem cell insult.

4. The method of claim 1, wherein the agent is an inhibitory nucleic acid molecule that decreases the expression of a P2Y14 receptor polynucleotide or polypeptide.

5. The method of claim 4, wherein the inhibitory nucleic acid molecule is an antisense molecule, short interfering RNA (siRNA), or short hairpin RNA (shRNA).

6. The method of claim 1, wherein the agent is an antibody that inhibits P2Y14 receptor activation.

7. The method of claim 6, wherein the antibody blocks the binding of a ligand to the receptor.

8. A method for treating blood cell injury in a subject, the method comprising contacting a hematopoietic stem cell of the subject with an effective amount of an agent that inhibits the expression or biological activity of a P2Y14 receptor in the subject following an insult to a hematopoietic stem cell, thereby treating blood cell injury in the subject.

9-16. (canceled)

17. A method for increasing the amount of blood cells in a subject in need thereof, the method comprising administering to the subject an effective amount of an agent that inhibits the expression or biological activity of a P2Y14 receptor, thereby increasing the amount of blood cells in the subject.

18-20. (canceled)

21. The method of claim 17, wherein the agent is an inhibitory nucleic acid molecule that decreases the expression of a P2Y14 receptor polynucleotide or polypeptide.

22. The method of claim 21, wherein the inhibitory nucleic acid molecule is an antisense molecule, short interfering RNA (siRNA), or short hairpin RNA (shRNA).

23. The method of claim 17, wherein the agent is an antibody that inhibits P2Y14 receptor.

24. (canceled)

25. The method of claim 17, wherein the agent is a small molecule.

26-27. (canceled)

28. A method of increasing stem cell survival, growth or proliferation the method comprising contacting a stem cell or progenitor cell that expresses a P2Y14 receptor with an effective amount of an agent that inhibits P2Y14 receptor expression or biological activity thereby increasing stem cell survival or proliferation.

29. The method of claim 1, wherein the stem cell is selected from the group consisting of a mesenchymal, skin, neural, intestinal, liver, cardiac, prostate, mammary, kidney, pancreatic, retinal and lung stem cell.

30-49. (canceled)

50. A method of increasing the number of self-renewing stem cells in a subject in need thereof, the method comprising the steps of:
contacting an isolated population of cells comprising stem cells with a P2Y14 receptor inhibitor; and
administering the cells to the subject, thereby increasing the amount of self-renewing stem cells in the subject.

51-59. (canceled)

60. A method of identifying a candidate compound that promotes stem cell survival, growth or proliferation, the method comprising:
a) contacting a cell that expresses a P2Y14 receptor with a candidate compound; and
b) detecting a decrease in OPN expression or activity, wherein the decrease identifies a candidate compound that promotes stem cell survival or proliferation.

61. (canceled)

62. An isolated bone marrow derived cell comprising a P2Y14 receptor inhibitory nucleic acid molecule, wherein the P2Y14 receptor inhibitory nucleic acid molecule reduces expression of the P2Y14 receptor in the cell.

63-64. (canceled)

65. A kit for promoting stem cell survival, growth, or proliferation comprising a P2Y14 receptor inhibitor, and instructions for using the inhibitor to promote stem cell survival, growth, or proliferation.

66. A kit for increasing stem cell expansion comprising an agent that inhibits the P2Y14 receptor expression of biological activity and instructions for using the agent to increase stem cell expansion.

67-68. (canceled)
